(19) United States
(12) Patent Application Publication
(10) Pub. No.: US 2026/0035661 A1
(43) Pub. Date: Feb. 5, 2026

(54) 3D CELL CULTURE METHODS

(71) Applicants: FONDAZIONE TELETHON ETS (50%), ROMA (IT); OSPEDALE SAN RAFFAELE S.R.L. (50%), Milan (IT)

(72) Inventors: Raffaella Di Micco, Milano (IT); Lucrezia Della Volpe, Milano (IT); Federico Midena, Milano (IT)

(73) Assignees: FONDAZIONE TELETHON ETS (50%), Roma (IT); OSPEDALE SAN RAFFAELE S.R.L. (50%), Milan (IT)

(21) Appl. No.: 19/119,233

(22) PCT Filed: Oct. 11, 2023
(86) PCT No.: PCT/IB2023/060206

§ 371 Date: Apr. 8, 2025

(30) Foreign Application Priority Data
Oct. 11, 2022 (EP) 22200888.0

(51) Int. Cl. C12N 5/0783; A61K 35/12; A61K 35/17; A61K 35/28; A61K 40/11; C12N 5/00; C12N 5/0775; C12N 5/0789; C12N 5/10; C12N 9/22; C12N 15/113; C12N 15/864; C12N 15/867

(52) U.S. Cl. CPC C12N 5/0636; A61K 35/17; A61K 35/28; A61K 40/11; C12N 5/0062; C12N 5/0647; C12N 5/0662; C12N 5/10; C12N 9/222; C12N 15/113; C12N 15/8645; C12N 15/867; A61K 2035/124; C12N 2310/20; C12N 2501/20; C12N 2510/00; C12N 2513/00; C12N 2740/10043; C12N 2740/15043; C12N 2750/14143; C12N 2799/027

(57) ABSTRACT

The present invention is directed to a method for ex-vivo-engineering of cells, in particular stem cells or T cells, preferably hematopoictic stem and/or progenitor cells, mesenchymal stem cells, or T cells comprising a step of culturing the cells on a three-dimensional scaffold. The method of the invention is capable of improving the efficiency of genetic modification of cells and the functionality of the engineered cells.

### FIELD OF THE INVENTION

The present invention is directed to a method for ex-vivo-engineering of cells, in particular stem cells or T cells, preferably hematopoietic stem and/or progenitor cells, mesenchymal stem cells, or T cells comprising a step of culturing the cells on a three-dimensional scaffold. The method of the invention is capable of improving the efficiency of genetic modification of cells and the functionality of the engineered cells.

### BACKGROUND

Gene correction approaches based on the transplantation of ex-vivo-engineered hematopoietic stem cells (HSCs) have shown promising results in the clinic. The concept of ex-vivo-engineering of stem cells is based on the genetic modification of a relatively small number of stem cells that are then transplanted in the receiving subject to undergo self-renewal and generate large numbers of genetically “corrected” progeny. This ensures a continuous supply of corrected cells for the rest of the patient's lifetime.

HSCs are particularly attractive targets for gene correction since their genetic modification will be passed to all the blood cell lineages as they differentiate. Furthermore, HSCs can be easily and safely obtained, for example from bone marrow, mobilized peripheral blood, and umbilical cord blood.

Recent ex vivo gene therapy clinical trials for patients affected by primary immunodeficiencies, hemoglobinopathies, or inborn errors of metabolism have shown high levels of gene transfer into hematopoictic stem and progenitor cells (HSPCs), which were stably maintained in multiple hematopoietic lineages; moreover, the post-transplant hematopoiesis reconstituted by polyclonal, gene-marked HSPCs has provided substantial and sustained therapeutic benefit to most treated patients to date. Still, genetic engineering to high levels and in large scale remains challenging. In particular, the side effects reported in these gene therapy trials are typically related to the conditioning regimen and include mucositis and temporary bone marrow (BM) aplasia. Also, prolonged neutropenia due to delayed engraftment of ex vivo modified cells has been observed.

The protocols for ex-vivo gene manipulation in cells have been tailored over the past decade to ensure high efficiency of gene manipulation and long-term gene modification. Also, preservation of the HSC long-term repopulating capacity upon gene manipulation is very important. For example, for gene correction to be efficacious, effective gene manipulation into target cells must be reached, without inducing detrimental effects on their biological properties.

Experimental evidence has been accumulated that cultured HSCs progressively lose engraftment potential, thus impeding their homing into the niche and driving lineage commitment and differentiation. Also, while in culture engineered stem cells can undergo biological damages, that end up in genome instability and loss of functionality of engineered cells.

Therefore, although progress has been made in improving gene modification protocols, there remains a need for further approaches to improve those protocols and achieve efficient, long-term genetic modification and maintenance of the HSC long-term repopulating capacity upon gene manipulation.

Particularly needed is the provision of improved methods for ex vivo gene editing in primary cells, and in particular in the primitive HSPC subset, as this is constrained by gene transfer efficiency and limited proficiency of homology directed DNA repair (HDR), likely due to HSC quiescence, to low levels of expression of the HDR machinery and conversely to high activity of the error-prone non homologous end joining (NHEJ) pathway. Thus, it is crucial to enhance the efficiency of gene editing in HSC while fully preserving the long-term repopulating activity of the cells. Also, in general there is the need for providing effective methods for ex vivo gene modification of cells, including for instance for ex vivo genetic modification of T cells to be used as medicament, e.g., for CAR-T gene therapy.

### BRIEF DESCRIPTION OF THE INVENTION

The limitations of the prior art are overcome by the present invention, providing a method for ex-vivo-engineering of cells, in particular of hematopoietic stem and/or progenitor cells, mesenchymal stem cells, or T cells, comprising culturing the cells on a three-dimensional scaffold, before, during and/or after genetically modifying the cells ex vivo.

In a first aspect, the present invention is thus directed to a method for ex-vivo-engineering of cells, preferably of stem cells or T cells, as set forth by the present claims.

The invention is also directed to the ex-vivo engineered cells obtained by said method and to their use, as such or in a pharmaceutical formulation, as a medicament, preferably in a method of treatment of a disease, more preferably of a genetic disease. The invention is best understood from the following detailed description when read in conjunction with the accompanying drawings.

### BRIEF DESCRIPTION OF FIGURES

FIG. 1 shows: A) Schematic representation of experimental design: cord or mobilized peripheral blood-derived HSPCs were thawed (I) and seeded (II) on standard plastic culture wells or on 3D nichoids scaffolds; after 72 hours, cells were transplanted into NSG mice (III), then analysed in vitro on days 3 and 7 in culture; B) Relative quantification of the percentage of HSPC subpopulation composition (from bottom to top of bars; CD34+CD133−; CD34+CD133+; and CD34+CD133+CD90+) on days 3 and 7 post thawing (n=3); C) Quantification of single and double-strand DNA breaks by alkaline comet assay on days 3 and 7 post thawing (n=1). More than 100 cells per condition were analysed. D) Quantification of γH2AX (left) and pRPA (right) foci on day 7 (n=1). More than 100 cells per condition were analysed; E) Number of colonies formed by HSPCs cultured on standard culture wells (plastic) or nichoids 72 or 168 h post-thawing (n=3). Statistical analysis on mixed colonies is shown. F) Percentage of human CD45+ cells (left) and graft composition (right) in the peripheral blood of NSG mice transplanted with 1.5×105 HSPCs after 3 days of culture on standard culture wells (plastic) or nichoids. G) Percentage of human CD45+ cells in the bone marrow of transplanted NSG mice. H) Percentage of human CD45+ cells in the spleen of transplanted NSG mice. Mean±SEM; (n=5). Mann-Whitney tests. *p<0.05; **p<0.01; ****p<0.0001.

FIG. 2 shows: A) Schematic representation of experimental design: cord or mobilized peripheral blood-derived HSPCs were thawed (I) and seeded (II) on standard plastic culture wells or on 3D nichoids scaffolds; cells were gene-edited (III) by electroporation of Cas9 RNPs and AAV6 on day 3 post-thawing, and re-seeded (IV) on standard culture wells. Subsequent in vitro analyses were performed at 24 or 96 h post-editing. B) Relative quantification of the percentage of HSPC subpopulation composition (CD34+CD133−; CD34+CD133+; and CD34+CD133+CD90+) (n=3). C) Percentage of gene-edited CD34+ cells (GFP+) assessed by flow cytometry (n=3). D) Proliferation rates of HSPCs cultured on standard plastic culture wells (grey dots) or on nichoids (black dots) counted at thawing. at the moment of gene-editing (GE), and 24 or 96 h post-editing. Fold increase compared to GE is reported on the graph (n=3). E) Quantification of single and double-strand DNA breaks by alkaline comet assay (n=1). More than 100 cells per condition were analysed. F) Number of colonies formed at 24 h and 96 h post-editing by gene-edited HSPCs cultured on standard culture wells (plastic) or nichoids (n=3). Statistical analysis on mixed colonies is shown. Mann-Whitney tests. **p<0.01.

FIG. 3 shows: A) Percentage of human CD45+ cells in the peripheral blood of NSG mice transplanted with 1.5×10⁵ cord blood-derived HSPCs 24 h post-editing upon standard culture well (plastic) or nichoid culture. B) Percentage of gene-edited (GFP+) cells in the peripheral blood of transplanted NSG mice. C) Percentage of human CD45+ cells in the bone marrow of transplanted NSG mice. D) Percentage of human CD45+cells in the spleen of transplanted NSG mice. Mean±SEM; (n=11). Mann-Whitney tests. *p<0.05; **p<0.01.

FIG. 4 shows: A) Schematic representation of experimental design: mobilized peripheral blood-derived HSPCs were thawed (I) and seeded (II) on standard culture wells (plastic) or nichoids and transduced (III) with a lentiviral vector (MOI 100 upon PGE2 pre-stimulation) at 24 h post-thawing. Subsequent analyses (IV) were performed at 14 h post-transduction. B) Percentage of gene-transferred CD34+ cells (GFP+) assessed by flow cytometry (n=3). C) Number of colonies formed at 24 h post-transduction by transduced HSPCs cultured on standard culture wells (plastic) or nichoids (n=3).

FIG. 5 shows: A) Schematic representation of experimental design: mobilized peripheral blood-derived HSPCs (mPB-derived CD34+ cells) were thawed (I) and seeded (II) in an expansion media on standard culture wells (plastic) or nichoids and transduced (III) with a lentiviral vector (MOI 30 upon CsH pre-stimulation) on day 3. Subsequent analyses (IV) were performed on day 8. B) Percentage of gene-transferred CD34+ cells (GFP+) from D assessed by flow cytometry (n=6). C) Number of colonies formed at day 8 by transduced HSPCs cultured on standard culture wells (plastic) or nichoids (n=3) from D. Statistical analysis was performed on the total number of colonies. Mann-Whitney tests. *p<0.05.

FIG. 6 shows: A) Schematic representation of experimental design: mobilized peripheral blood-derived HSPCs were thawed (I) and seeded (II) on a cell culture bag or nichoids. On day 3. part of the cells were collected for subsequent analyses (IIIa) and part were gene-edited (IIIb) by electroporation of Cas9 RNPs and AAV6, and re-seeded on standard culture wells (plastic). Further analyses (IV) were performed at 24 or 96 h post-editing. B) Percentage of gene-edited CD34+ cells (GFP+) assessed by flow cytometry (n=1). C) Cell viability assessed by flow cytometry (n=1). D) Number of colonies formed by non-edited HSPCs cultured on cell culture bag or nichoids on day 3 and by gene-edited HSPCs at 24 h post-editing (n=1, 3 technical replicates).

### DETAILED DESCRIPTION OF THE INVENTION

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a cell” includes a plurality of such cells.

The term “individual” or “subject” herein refers to a mammal, preferably human or non-human mammal, more preferably mouse, rat, other rodents, rabbit, dog, cat, pig, cow, horse, or primate, further more preferably human.

The term “pharmaceutically acceptable excipient” refers to a non-toxic solid, semisolid, or liquid filler, diluent, encapsulating material, or formulation auxiliary of any conventional type that may optionally be included in the compositions of the invention and that causes no significant adverse toxicological effects to the patient. A pharmaceutically acceptable excipient is essentially non-toxic to recipients at the employed dosages and concentrations and is compatible with other ingredients of the formulation. The number and the nature of the pharmaceutically acceptable excipients depend on the desired administration form.

The term “vector” refers to a particle capable of delivering, and optionally expressing, one or more polynucleotides of interest into a host cell. Examples of vectors include, but are not limited to, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. The vector can be a cloning vector, suitable for propagation and for obtaining polynucleotides, gene constructs or expression vectors incorporated to several heterologous organisms. A vector is capable of transferring nucleic acid sequences to target cells, therefore also viral vectors, non-viral vectors, particulate carriers, and liposomes are included in the term “vector”. Typically, “vector construct”, “expression vector” and “gene transfer vector” mean any nucleic acid construct capable of directing the expression of a nucleic acid of interest and which can transfer nucleic acid sequences to target cells. Thus, the term includes cloning and expression vehicles, as well as viral vectors.

The term “recombinant plasmid” or “plasmid” refers to a small, circular, double-stranded, self-replicating DNA molecule obtained through genetic engineering techniques capable of transferring genetic material of interest to a cell, which results in production of the product encoded by that said genetic material (e.g., a protein polypeptide, peptide or functional RNA) in the target cell.

Furthermore, the term “recombinant plasmid” or “plasmid” also refers to a small, circular, double-stranded, self-replicating DNA molecule obtained through genetic engineering techniques used during the manufacturing of viral vectors as carriers of the recombinant vector genome.

Non-viral delivery systems include but are not limited to DNA transfection methods. Here, transfection includes a process using a non-viral vector to deliver a polynucleotide to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nature Biotechnology (1996) 14:556) and combinations thereof.

The term “recombinant viral vector” or “viral vector” refers to an agent obtained from a naturally-occurring virus through genetic engineering techniques capable of transferring genetic material (e.g., DNA or RNA) of interest to a cell, which results in production of the product encoded by that said genetic material (e.g., a protein polypeptide, peptide or functional RNA) in the target cell. Herein, the terms “vector transgene” or “recombinant vector transgene” refer to a transgene that is transferred to the recipient cell upon transduction.

The term “viral vector” or “recombinant viral vector”, as used herein, also refers to the recombinant viral particles being a packaged viral vector, capable of binding to and entering recipient cells, delivering the vector transgene.

Viral delivery systems include but are not limited to adenoviral vectors, adeno-associated viral (AAV) vectors, herpes viral vectors, retroviral vectors, lentiviral vectors and baculoviral vectors.

The terms “engineered” or “genetically modified”, as referred to cells, are herein used interchangeably.

“Engineering” or “genetic modification” or “genetic manipulation” of a cell according to the present invention include “gene transfer”, i.e., addition of a copy of a gene into the genome of the cell, such as a correct copy of a gene that is completely or partially deleted or completely or partially not functional in the isolated cell (gene therapy). In particular, the term “gene transfer” refers to the transfer of genetic material (e.g., DNA or RNA) of interest into a cell to treat or prevent a genetic or acquired disease or condition. The genetic material of interest encodes a product (e.g., a protein polypeptide, peptide or functional RNA) whose production in vivo is desired. For example, the genetic material of interest can encode an enzyme, hormone, receptor, or polypeptide of therapeutic value. “Gene transfer” or “gene delivery” also refer to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells.

“Engineering” or “genetic modification” or “genetic manipulation” of a cell according to the present invention also include “gene editing”, i.e., modification of the genome of the cell at a specific location to correct or alter a genetic sequence. The term “gene editing” refers to a type of genetic engineering in which a nucleic acid is inserted, deleted or replaced in a cell. The term “gene editing” thus encompasses targeted disruption of a gene coding sequence, precise sequence substitution for in situ correction of mutations and targeted transgene insertion into a predetermined locus.

In general, “engineering” or “genetic modification” or “genetic manipulation” of a cell comprise transduction, transfection and transformation methods.

Transfection is the process of introducing nucleic acid into host cells by non-viral methods. Transduction is the process of introducing foreign DNA or RNA into host cells through viral vectors.

“Host cells,” “cells”, “cell lines,” “cell cultures”, “engineered cells” and other such terms denoting microorganisms or higher eukaryotic cell lines cultured as unicellular entities refer to cells which can be, or have been, used as recipients for recombinant vector or other transferred DNA, and include the original progeny of the original cell.

The terms “culture or culturing”, “growth or growing”, referred to cells are used herein interchangeably and are meant to indicate maintenance of a cell population in vitro or ex vivo, preferably including expansion of the cell population.

Cells may undergo increased apoptosis following transduction or transfection with a vector during cell culture. Cell survival may be readily analysed by the skilled person. For example, the numbers of live, dead and/or apoptotic cells in a cell culture may be quantified at the beginning of culture and/or following culture for a period of time (e.g., about 6 or 12 hours, or 1, 2, 3, 4, 5, 6, 7 or more days; preferably, the period of time begins with the transduction of the cells with a vector). As an example, the effect of an agent or method of culture on cell survival may be assessed by comparing the numbers and/or percentages of live, dead and/or apoptotic cells at the beginning and/or end of the culture period between experiments carried out in the presence and absence of the agent, but under otherwise substantially identical conditions. Cell numbers and/or percentages in certain states (e.g., live, dead or apoptotic cells) may be quantified using any of a number of methods known in the art, including use of haemocytometers, automated cell counters, flow cytometers and fluorescence activated cell sorting machines. These techniques may enable distinguishing between live, dead and/or apoptotic cells. In addition, or in the alternative, apoptotic cells may be detected using readily available apoptosis assays (e.g., assays based on the detection of phosphatidylserine (PS) on the cell membrane surface, such as through use of Annexin V, which binds to exposed PS; apoptotic cells may be quantified through use of fluorescently-labelled Annexin V), which may be used to complement other techniques.

The term “engraftment” as used herein refers to the ability of cells to populate and survive in a subject following their transplantation, i.e., in the short and/or long term after transplantation.

For example, engraftment may refer to the number and/or percentages of haematopoietic cells descended from transplanted haematopoietic stem and/or progenitor cells (e.g., graft-derived cells) that are detected about 1 day to 24 weeks, 1 day to 10 weeks, or 1-30 days or 10-30 days after transplantation. In a xenograft model of human haematopoietic stem and/or progenitor cell engraftment and repopulation, engraftment may be evaluated in the peripheral blood as the percentage of cells deriving from the human xenograft (e.g., positive for the CD45 surface marker), for example. Engraftment may be readily analysed by the skilled person. For example, the transplanted haematopoietic stem and/or progenitor cells may be engineered to comprise a marker (e.g., a reporter protein, such as a fluorescent protein), which can be used to quantify the graft-derived cells. Samples for analysis may be extracted from relevant tissues and analysed ex vivo (e.g., using flow cytometry).

The terms “scaffold”, “matrix” or “three-dimensional, or 3D, scaffold or matrix” are used herein to indicate a three-dimensional support for growing cells.

The term “two-dimensional” cell culture refers to the seeding of cells within a petri dish or housing cells in a flask or bag, in a static liquid culture.

The present invention is directed to a method for ex-vivo-engineering of cells, in particular stem cells, preferably hematopoietic stem and/or progenitor cells (HSCs/HSPCs) or mesenchymal stem cells, or T cells, comprising culturing the cells on a three-dimensional support, before, during and/or after genetic modification of the cells ex vivo.

Preferably, the step of culturing the cells on a three-dimensional scaffold is carried out for 1 to 14 days, more preferably for 1 to 10 days, most preferably for 1 to 8 days. Preferably, the cells are cultured on a three-dimensional scaffold for at least 1, at least 2, at least 3, at least 4, at least 5, at least 6, at least 7, at least 8, or at least 9 days, before being collected or being transferred on a different culture support (such as a 2d culture support, a flask, or a bag).

Preferably, genetic modification ex vivo is carried out on cells that have been cultured on a three-dimensional scaffold for at least 1, at least 2, at least 3 days, or at least 4 days. Optionally, genetic modification ex vivo is carried out on cells maintained in culture on the three-dimensional scaffold.

According to preferred aspects of the present invention, genetic modification of the cells is gene transfer, such as gene transfer for gene therapy, and/or gene editing.

Genetic modification preferably comprises, or consists of, transduction of a viral vector in a cell, more preferably an adeno-associated vector (AAV) or a retroviral vector, most preferably a lentiviral vector, or an integration-defective lentiviral vector (IDLV).

In some embodiments, genetic modification includes transduction of cells with RNA vectors, for example, using liposomes or lipid nanoparticles. In some embodiments, the RNA vector is in the form of a liposome or lipid nanoparticle.

Preferably, the ex vivo genetic modification according to the present invention is gene editing.

Gene editing may be achieved using engineered nucleases, which may be targeted to a desired site in a polynucleotide (e.g., a genome).

Gene editing is in fact based on the design of artificial endonucleases that target a double-strand break (DSB) or nick into the sequence of interest in the genome. Cells repair the DSB through two major mechanisms, Non-Homologous End-Joining (NHEJ) or Homology Directed Repair

(HDR), although other repair mechanisms might be eventually exploited. NHEJ often creates small insertions or deletions (“indels”) at the target site that may disrupt the coding sequence of a gene, whereas HDR can be exploited to precisely introduce a novel sequence at the target site by providing an exogenous template DNA bearing homology to the sequences flanking the DSB. Multiple platforms of artificial endonucleases can be used to target a locus of interest, including Zinc Finger Nucleases (ZFNs), TAL effector nucleases (TALENs) and RNA-based CRISPR/Cas9 nucleases or similar.

Such nucleases may be delivered to a target cell using vectors, such as viral or non-viral vectors. Examples of suitable nucleases known in the art include zinc finger nucleases (ZFNs), transcription activator like effector nucleases (TALENs), and the clustered regularly interspaced short palindromic repeats (CRISPR)/Cas system (Gaj, T. et al. (2013) Trends Biotechnol. 31: 397-405; Sander, J.D. et al. (2014) Nat. Biotechnol. 32: 347-55). Meganucleases (Silve, G. et al. (2011) Cur. Gene Ther. 11: 11-27) may also be employed as suitable nucleases for gene editing.

“CRISPR/Cas system” refers collectively to transcripts and other elements involved in the expression of, or directing the activity of, CRISPR-associated (“Cas”) genes, including sequences encoding a Cas gene and a guide RNA, wherein the guide RNA (gRNA) may be selected to enable a Cas domain to be targeted to a specific sequence (van der Oost et al. (2014) Nat. Rev. Microbiol. 12: 479-92). Methods for the design of gRNAs are known in the art. Furthermore, fully orthogonal Cas9 proteins, as well as Cas9/gRNA ribonucleoprotein complexes and modifications of the gRNA structure/composition to bind different proteins, have been recently developed to simultaneously and directionally target different effector domains to desired genomic sites of the cells (Esvelt et al. (2013) Nat. Methods 10: 1116-21;Zetsche, B. et al. (2015) Cell pii: S0092-8674 (15) 01200-3; Dahlman, J.E. et al. (2015) Nat. Biotechnol. 2015 Oct. 5. doi: 10.1038/nbt.3390. [Epub ahead of print]; Zalatan, J.G. et al. (2015) Cell 160: 339-50; Paix, A. et al. (2015) Genetics 201: 47-54), and are suitable for use in the invention.

Preferably, the gene editing according to preferred aspects of the present invention comprises the use of one or more zinc-finger nucleases, transcription activator like effector nucleases

(TALENs) and/or CRISPR system.

In general, a CRISPR system is characterized by elements that promote the formation of a CRISPR complex at the site of a target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. The target sequence may comprise any polynucleotide, such as DNA or RNA polynucleotides. In some embodiments, the target sequence is located in the nucleus or cytoplasm of the cell. In some embodiments, the target sequence may be within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

According to preferred embodiments the cells in the method of the invention are genetically modified by transduction or transfection of with one or more vectors encoding one or more effectors of the genetic modification, such as transgenes, nucleases, Cas nuclease, gRNAs, etc.

Preferably, the CRISPR system includes a non-coding RNA molecule (guide RNA, or gRNA), which sequence-specifically binds to DNA, and a Cas protein (e.g., Cas9), with nuclease functionality (e.g., two nuclease domains).

Preferably, in gene editing of the cells of the present invention, a Cas nuclease and gRNA are introduced into the cell to be engineered.

According to the present invention, a three-dimensional (3D) scaffold can be any known scaffold in the art, such as a hydrogel, a membrane (or tube), a 3D matrix, synthetic or natural.

Materials such as metals, glasses and ceramics can constitute a 3D scaffold, as well as polymers, synthetic or natural derived. Different kinds of polymer can be used to form 3D scaffolds, ranging from inert to biodegradable (polyester, polyethylene glycol, polyamide, polyglycolic acid, polylactic acid).

Hydrogels typically comprise water and natural biomolecules such as alginate, gelatine, hyaluronic acid, agarose, laminin, collagen or fibrin.

Non-gel polymer scaffolds commonly comprise natural polymers such as collagen, fibrin, alginate, silk, hyaluronic acid, and chitosan. As for synthetic polymers, there is poly(lactic acid) (PLA), poly(glycolic acid) (PGA), and polycaprolactone (PCL). Composites can also be used to build scaffolds, made of two or more distinctly different materials (ceramics combined with polymers for instance) developed to takes advantages of both materials properties to meet mechanical and physiological requirements.

Particularly preferred as 3D scaffold according to the method of the invention is a matrix as disclosed by Raimondi et al. 2014 or a supermatrix as described in WO2017037108, the content of which is incorporated herein by reference; briefly, said super matrix, also called “nichoids” comprises at least two matrices of synthetic niches, wherein each matrix comprises n×m synthetic niches, wherein n and m, the same or different from each other, have a value≥1, provided that one of m or n is ≥2 and with a maximum value of m and n which allows to maintain the structure of the single synthetic niche intact such that shrinking of the material does not cause significant disruptions, and wherein the distance (d) between a synthetic niche matrix and the other is greater than zero, and wherein in each matrix every synthetic niche has one or more walls in common with the other synthetic niche(s) of the matrix. The supermatrix according to the invention is obtained using the two-photon laser polymerization (2PP) technique.

The dimensions of the single niche may vary according to the specific type of cell being cultured, while always maintaining three-dimensional structure.

For example, the width and depth of the niche may vary between 20 and 500 μm with pores that vary from 5 to 100 μm, preferably from 10 μm to 30 μm. Height may vary from 5 to 500 μm, preferably from 30 μm to 100 μm. Each niche can have several layers of lattices, for example from 2 to 10 layers, preferably from 4 to 6 layers. Confinement walls are preferably made of parallel rods at a distance one from the other which may vary, for example, from 2 to 30 μm, preferably from 2 to 10 μm, more preferably it is 5 μm.

In case highly proliferative stem cells are cultured, such as pluripotent stem cells (a height of more than 30 μm is preferable.

The material utilized for production is a resin. Preferably, it is a photopolymerizable resin.

Also preferred as 3D scaffold according to the invention is a silk-based 3D scaffold, such as the silk sponge described by Di Buduo et al., Biomaterials, Volume 146, 2017, pages 60-71 and in WO2021113830, the content of which is incorporated herein by reference.

Briefly silk sponges can be obtained with salt leaching methods and preferably functionalized with components of the natural extracellular matrix of the cultured cell.

Optionally, the 3D scaffold can be or be connected to an implant, such as a device comprising separate chambers each comprising a 3D scaffold.

Surprisingly, growing on a 3D scaffolds the cells that will be and/or have been engineered, preferably by gene transfer or gene editing, improves effectiveness of the ex vivo engineering method, not only by helping stem cells in maintaining their staminality, but also improving survival of cells, reducing DNA damage upon genetic manipulation, and/or improving preservation of cells biological properties and of cells in vivo functionality, such as engrafting and clonogenic efficiency of engineered stem cells.

Preferably, the cells that undergo genetic modification in the method of the present invention are haematopoietic stem and/or progenitor cells. More preferably, the cells comprise CD34+ cells, and/or CD34+CD133+ cells, most preferably CD34+CD133+CD90+ cells.

Preferably, the cells are human cells.

Preferably, the cells are mobilised peripheral blood HSPCs, cord blood cells or bone marrow cells.

The method of the invention preferably comprises the steps of: providing isolated cells, more preferably isolated hematopoietic stem and progenitor cells (HSPCs), T cells, or mesenchymal stem cells (MSCs), and genetically modifying said cells, obtaining an ex vivo engineered cell population, the method being characterized in that it comprises culturing cells on a three dimensional scaffold before, during and/or after the step(s) of genetically modifying the cells.

Preferably, the genetic modification comprises, or consists of, transfecting and/or transducing the isolated cells; more preferably, the transduction includes stimulation of cells in the presence of a human cytokine mix (preferably IL-6. TPO, SCF, and FLT3-1), more preferably for about 22-hour, followed by the addition of the viral particles, preferably for about 14 hours upon transduction.

The ex vivo engineered cells obtained at the end of the method of the invention are preferably resuspended in a freezing medium and frozen until used.

Preferably the cells are autologous cell, i.e., cells obtained from a subject, to which the cells are reinfused, once they are genetically modified.

The invention is also directed to a method of expanding ex vivo the cells isolated from a subject affected by a disease, such as a genetic disease, said method comprising providing isolated cells, preferably isolated hematopoietic stem and/or progenitor cells, T cells, or mesenchymal stem cells, preferably said cells bearing a genetic defect, from a subject affected by a disease and culturing the cells on a 3D scaffold. More preferably, the cells are subjected to genetic modification.

According to the present invention, the cells are preferably CD34+ HSPCs obtained and isolated by leukapheresis (after mobilization by mobilizing agents such as G-CSF and Plerixafor) or bone marrow harvest (for subjects unsuitable for mobilization/leukapheresis); cells are then preferably purified by means of immunomagnetic beads, to obtain highly pure CD34+ cells.

Preferably, the method of the invention comprises seeding the cells at a concentration of 1×10⁵ cells/ml to 10×10⁵ cells/ml, e.g. about 2×10⁵ cells/ml, or about 5×10⁵ cells/ml.

According to preferred embodiments, the method of the invention comprises: on day 0, seeding cells, preferably CD34+ HSPCs, on a three dimensional scaffold and stimulating the cells with cytokines for a suitable time, preferably for 22±2 hours; on day 1, genetically modifying the cells, preferably by transducing cells with a viral vector, preferably in the same culture medium (1-hit transduction protocol); after transduction, collecting the genetically modified cells. Optionally the cells are genetically modified by transfection and collected after transfection. Preferably the genetically modified cells are the washed and resuspended, more preferably at a concentration of 2.5-10×10⁶ cells/ml, in a minimum volume of freezing medium (e.g., 20 ml), and cryopreserved under vapor of liquid nitrogen in cryobags.

In some embodiments, after genetical modification, the cells are maintained in culture, or re-seeded, on a three dimensional support and grown for one or more days before collection.

Preferably, the cells are maintained in culture on the 3D scaffold for 1 to 8 days after genetic modification, more preferably for 2 to 6 days after genetic modification, most preferably for 3 to 5 days after genetic modification, or for about 4 days after genetic modification of cells.

In an alternative preferred embodiment of the method described above, on day 0 the cells are cultured in a 2D cell culture and only seeded on a 3D scaffold after genetic modification.

Preferably, at least one viral transduction enhancer, more preferably PGE, CsH, CsA or a poloxamer, and/or at least one expansion enhancer, more preferably UM171, UM729, StemRegenin1 (SR1), diethylaminobenzaldehyde (DEAB), LG1506, BIO (GSK3β inhibitor), NR-101, trichostatin A (TSA), garcinol (GAR), valproic acid (VPA), copper chelator, tetraethylenepentamine, and nicotinamide, is added to the cell culture before transduction according to optimized protocols, for instance as described in WO2013049615,WO2018193118, WO2013127964, WO2023066735 and in Delville et al.

Preferably, the at least one expansion enhancer is added to the cells after seeding and it is maintained in contact with the cells for at least 1, at least 2, at least 3 days, before genetic modification of the cells.

Preferably, at least one viral transduction enhancer is added to the cells at least 1, at least 2, or at least 3 hours, before genetic modification of the cells.

Any suitable concentration of expansion enhancer may be used, for example as described in Huang. X., et al., 2019 (F1000Research, 8, 1833). In some embodiments, the culture medium comprises UM171 or UM729. The concentration of UM171 may be about 10-200 nM, about 20-100 nM, or about 35 nM. In some embodiments, the culture medium comprises SR1. The concentration of 35 SR1 may be about 0.1-10 μM, about 0.5-5 μM, or about 1 μM. In some embodiments, the P124913IT 37 culture medium comprises UM171 (e.g., in a concentration of about 35 nM) and SR1 (e.g., in a concentration of about 1 μM). In some embodiments, the culture medium comprises SCF (e.g., in a concentration of about 300 ng/ml), FLT3-L (e.g., in a concentration of about 300 ng/ml), TPO (e.g., in a concentration 5 of about 100 ng/ml), UM171 (e.g., in a concentration of about 35 nM) and SR1 (e.g., in a concentration of about 1 μM). In some embodiments, the culture medium comprises SCF (e.g., in a concentration of about 300 ng/ml), FLT3-L (e.g., in a concentration of about 300 ng/ml), TPO (e.g., in a concentration of about 100 ng/ml), UM171 (e.g., in a concentration of about 35 nM), SR1 (e.g., in a 10 concentration of about 1 μM), and PGE2 (e.g., in a concentration of about 10 μM).

According to preferred embodiments of the method of the invention, the cells, more preferably HSPCs, are genetically engineered to express an engraftment enhancer. More preferably said engraftment enhancer is CD47 and/or C-X-C chemokine receptor type 4 (CXCR4). More preferably, the cells are transduced or transfected with one or more vectors encoding the CD47 and/or CXCR4; the CD47 and CXCR4 may be, for example, encoded on separate vectors or on the same vector. The vector can be a plasmid or a viral vector, for example a retroviral, adenoviral or adeno-associated viral vector. Preferably, CD47 and/or CXCR4 arc overexpressed in the cell. Optionally, RNA encoding the CD47 and/or CXCR4 is introduced into the cells using RNA electroporation, or CD47 and/or CXCR4 protein is directly introduced into the cells, for example using protein electroporation.

According to preferred embodiments, the method of the invention also comprises culturing the cells in the presence of an inhibitor of senescence, such as inhibitor of MAPK/ERK signaling, an IL-1 inhibitor and/or an NF-κB inhibitor. Preferably, the inhibitor of MAPK/ERK signaling is a MAP3K inhibitor, a MAK2K inhibitor, a MAPK inhibitor, preferably an MKK7 inhibitor, an MKK4 inhibitor, an MKK3/6 inhibitor, an MEK1/2 inhibitor, a JNK inhibitor, a p38 inhibitor, a p53 inhibitor or an ERK inhibitor. Preferably, the inhibitor of p53 activation is a p53 dominant negative peptide, an ataxia telangiectasia mutated (ATM) kinase inhibitor or an ataxia telangiectasia and Rad3-related protein (ATR) inhibitor. In some embodiments, the inhibitor of p53 activation is pifithrin-α or a derivative thereof; KU-55933 or a derivative thereof; GSE56 or a variant thereof; KU-60019, BEZ235, wortmannin, CP-466722, Torin 2, CGK 733, KU-559403, AZD6738 or derivatives thereof; or an siRNA, shRNA, miRNA or antisense DNA/RNA, preferably wherein the inhibitor of p53 activation is GSE56 or a variant thereof. Without being bound to theory, inhibitors of senescence suitably inhibit DDR-dependent inflammation, thus (further) increasing the survival and/or engraftment of cells, in particular of haematopoietic stem cells, haematopoietic progenitor cells and/or T cells. Also, the inhibition of DDR-dependent inflammation (further) increases the efficiency of gene editing of haematopoietic cells, haematopoietic stem cells, haematopoietic progenitor cells, and/or T cells.

Preferably, the inhibitor(s) of senescence are added to the cell culture and maintained in contact with the cells for about 12-60 hours, 24-60 hours, 36-60 hours, or 42-54 hours, before the step of genetically modifying the cells.

Suitably, the inhibitor(s) may be active during genetic modification of cells.

Preferably, the one or more inhibitor(s) of senescence (e.g. the MAPK inhibitor, IL-1 inhibitor and/or NF-KB inhibitor, preferably the IL-1 inhibitor and/or NF-KB inhibitor) is added to the cells (e.g. in an in vitro or ex vivo culture) at a concentration of 0.5-200, 0.5-150, 1-100, 1-50, 5-30, 5-20 or 5-15 μM.

According to preferred embodiments, the ex vivo method of the invention comprises the following steps, in sequence:
seeding the cells on a three-dimensional (3D) scaffold and maintaining the same in culture on the 3D scaffold for at least 1, 2, 3 or 4 days before genetically modifying the cells; and
genetically modifying the cells ex vivo.

Optionally, the step of genetically modifying the cells ex vivo is carried out on cells on the 3D scaffold.

Preferably the cells are maintained in culture on the 3D scaffold in the presence of one or more of: senescence inhibitor(s), cytokine(s), viral transduction enhancer(s), expansion enhancer(s) or mixtures thereof.

Preferably, the cells are maintained in contact with at least one expansion enhancer for at least 1, at least 2, or at least 3 days, before being genetically modified. For instance the cells can be cultured in the presence of at least one expansion enhancer for 1 to 3 days, preferably for 2 to 3 days, before genetic modification of the same.

Preferably, the cells are maintained in contact with at least one transduction enhancer for at least 1, at least 2, or at least 3 hours, before being genetically modified. For instance the cells can be cultured in the presence of at least one expansion enhancer for 1 to 3 hours, preferably for 2 to 3 hours, before genetic modification of the same.

According to preferred embodiments, the step of culturing the cells on a three-dimensional (3D) scaffold, comprises adding and maintaining the cells in contact with at least one expansion enhancer for at least at least 1, at least 2, or at least 3 days, followed by adding and maintaining the cells in contact with at least one transduction enhancer(s), at least 3, at least 2, or at least 1 hour(s), before the step of genetically modifying the cells.

Preferably, the step of genetically modifying the cells ex vivo comprises or consists of transducing the cells with a vector, preferably a viral vector, more preferably for a period of time of 10 to 20 hours, for 12 to 16 hours, for 13 to15 hours, or for about 14 hours.

Optionally the step of genetically modifying the cells comprises two hits of transduction of the cells with a viral vector.

Preferably, transduction is carried out in the presence of at least one transduction enhancer(s).

Therefore, in preferred embodiments, the cells are maintained in contact with the at least one transduction enhancer(s) for up to 24 hours, up to 22 hours, up to 20 hours, up to 18 hours, up to 16 hours, or up to 14 hours.

Preferably, the method of the invention further comprises a step of washing out any means for genetically modifying the cells, such as viral vectors, at the end of the step of genetically modifying the cells.

The step of genetically modifying the cells, when being carried out by transducing the cells with a viral vector, preferably comprises contacting the cells with the viral vector for 10 to 20 hours, for 12 to 18 hours, for 14 to 16 hours.

Preferably, the method of the invention further comprises a step of collecting the genetically modified cells; more preferably, said further step is carried out at least 1, at least 2, at least 3, at least 4, or at least days after genetic modification of cells.

In particularly preferred embodiments of the method of the invention, the cells are seeded on a 3D scaffold and cultured thereon in a serum-free medium, supplemented with suitable nutrients and/or antibiotics. After 20 to 24 hours, at least one transduction enhancer is added to the medium. The cells are then transduced with a viral vector, for genetic modification of the same, and the vector is maintained in contact with the cells for 10 to 18 hours, more preferably for 12 to 16 hours, most preferably for about 14 hours. After said period of time, the viral vector is washed out. Preferably the engineered cells are immediately collected or frozen.

According to further preferred embodiments of the method of the invention, the cells are seeded on a 3D scaffold and cultured thereon in a serum-free medium, supplemented with suitable nutrients and/or antibiotics and further supplemented with at least one expansion enhancer.

Preferably, the expansion enhancer is maintained in contact with the cells for 1 to 3 days, more preferably for 2 to 3 days. Optionally, at least one transduction enhancer is then added to the medium. The cells are then transduced with a viral vector, for genetic modification of the same, and the vector is maintained in contact with the cells for 10 to 18 hours, more preferably for 12 to 16 hours, most preferably for about 14 hours. After said period of time, the viral vector is washed out. Preferably, the engineered cells are maintained in culture, more preferably on the 3D scaffold, for 1 to 4 days after the viral vector's wash out, before being collected or frozen.

The present invention further provides the engineered cells obtained by the method of the invention and to pharmaceutical formulations comprising the population of engineered cells of the invention and pharmaceutically acceptable carriers, diluents or excipients.

The invention may be useful in the treatment of the disorders listed in WO 1998/005635. For case of reference, part of that list is now provided: cancer, inflammation or inflammatory disease, dermatological disorders, fever, cardiovascular effects, hemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumor growth, invasion and spread, angiogenesis, metastases, malignant, ascites and malignant pleural effusion; cerebral ischemia, ischemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the invention may be useful in the treatment of the disorders listed in WO 1998/007859. For case of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumor immunity); regulation of hematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilizing specific cell types to sites of injury or infection); hemostatic and thrombolytic activity (e.g. for treating hemophilia and stroke); anti-inflammatory activity (for treating e.g. septic shock or Crohn's disease); as antimicrobials; modulators of e.g. metabolism or behavior; as analgesics; treating specific deficiency disorders; in treatment of e.g. psoriasis, in human or veterinary medicine. In addition, or in the alternative, the invention may be useful in the treatment of the disorders listed in WO 1998/009985. For case of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated of receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular discases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The applicability of the invention to T cells facilitates its application also in methods of ex vivo cell therapies that are based on infusion of modified T cells into patients, including anti-cancer strategies (such as using engineered CAR-T cells) and approaches based on infusion of universal donor T cells.

Therefore, in another aspect, the invention provides a population of ex vivo engineered cells, preferably stem cells or T cells, more preferably HSCs or HSPCs or mesenchymal stem cells, obtained by the method of the invention, or a pharmaceutical formulation thereof, for use as a medicament.

Preferably, the invention provides a population of ex vivo engineered cells, more preferably stem cells or T cells, most preferably HSCs or HSPCs or mesenchymal stem cells, obtained by the method of the invention, or a pharmaceutical formulation thereof for use in the treatment or prevention of a disease selected from: cancer, an immune disorder, a bacterial or viral infection, a genetic disease, blood diseases, β-thalassemia, Fanconi anemia, bone marrow failures disease, sickle cell disease, osteopetrosis, chronic granulomatous disease, metachromatic leukodystrophy, mucopolysaccharidoses disorders and other lysosomal storage disorders.

In preferred embodiments, the ex vivo engineered cells are administered as part of an autologous stem cell transplant procedure.

In other preferred embodiments, the ex vivo engineered cells are administered as part of an allogeneic stem cell transplant procedure.

Preferably, the subject receiving the cells is subjected to a mild myeloablative conditioning regimen or to non-myeloablative conditioning regimen a before administration of the cells.

It should be understood that all the possible combinations of the preferred aspects of the present invention are also described, and therefore similarly preferred.

Examples of preferred embodiments of the present invention and analyses of their efficacy are provided below for illustrative and non-limiting purposes.

### EXAMPLES

### Materials and Methods

Vectors and nucleases. AAV6 DNA donor templates were generated from a construct containing AAV2 inverted terminal repeats, produced by a triple-transfection method and purified by ultracentrifugation on a cesium chloride gradient. Design of the AAV6 donor templates carrying homologies for AAVS1 encompassing a PGK.GFP reporter cassette was previously reported (Schiroli, G. et al., 2019, Cell Stem Cell 24: 551-565). The sequence of the gRNA was designed using an online tool54 and selected for predicted specificity score and on-target activity. Genomic sequence recognized by the gRNA was previously reported (Schiroli, G. et al., 2019, Cell Stem Cell 24: 551-565). RNP complexes were assembled by incubating at a 1:1.5 molar ratio Streptococcus pyogenes (Sp)Cas9 protein (Aldevron) with pre-annealed synthetic Alt-R crRNA:tracrRNA (Integrated DNA Technologies) for 10 min at 25° C. together with 0.1 nmol of Alt-R Cas9 Electroporation Enhancer (Integrated DNA Technologies) added before electroporation according to the manufacturer's instructions. Lentiviral vectors encoding for a PGK.GFP reporter cassette were produced by transient transfection in 293T cells and were all VSV-g pseudotyped and concentrated by ultracentrifugation as previously described (Montini et al., 2006).

Vector maps were designed with SnapGene software v.5.0.7 (from GSL Biotech, available at snapgene.com) or Vector NTI Express v.1.6.2 (from Thermo Fisher Scientific, available at thermofisher.com).

Primary cells. Cord Blood (CB) CD34+ HSPCs were purchased frozen from Lonza and were seeded at the concentration of 5×10⁵ cells per ml in serum-free StemSpan medium (StemCell Technologies) supplemented with 100 IU ml-1 penicillin, 100 μg ml-1 streptomycin, 2% glutamine, 100 ng ml-1 hSCF (PeproTech), 100 ng ml-1 hFlt3-L (PeproTech), 20 ng ml-1 hTPO (PeproTech) and 20 ng ml-1 hIL-6 (PeproTech) and 10 μM PGE2 (at the beginning of the culture, Cayman). Culture medium was also supplemented with 1 μM SR1 (Biovision) and 50 nM UM171 (STEMCell Technologies), unless otherwise specified.

G-CSF mobilized peripheral blood (mPB) CD34+ HSPCs were purified with the CliniMACS CD34 Reagent System (Miltenyi Biotec) from Mobilized Leukopak (AllCells) according to the manufacturer's instructions. HSPCs were seeded at the concentration of 5×10⁵ cells per ml in serum-free StemSpan medium (StemCell Technologies) supplemented with 100 IU ml-1 penicillin. 100 μg ml-1 streptomycin, 2% glutamine, 300 ng ml-1 hSCF, 300 ng ml-1 hFlt3-L, 100 ng ml-1 hTPO and 10 μM PGE2 (at the beginning of the culture, Cayman). Culture medium was also supplemented with 1 μM SR1 and 35 nM UM171. For lentiviral transduction (38 h protocol), serum-free StemSpan medium (StemCell Technologies) was supplemented with 100 IU/ml-1 penicillin, 100 μg/ml-1 streptomycin, 2% glutamine, 300 ng ml-1 hSCF, 300 ng ml-1 hFlt3-L, 100 ng ml-1 hTPO, and 60 ng ml-1 hIL-3 (PeproTech); 10 μM PGE2 (Cayman) was added 2h before transduction. For the expansion experiment, 8 μM cyclosporin H (Sigma-Alrich) was added immediately before transduction. MACS GMP Cell Expansion Bags were purchased from Miltenyi Biotec.

All cells were cultured in a 5% CO₂ humidified atmosphere at 37° C.

Nichoids. Nichoids were manufactured as described by Ricci D. et al. (2017, Materials 10:1).

Cell detachment from nichoids. Supernatant suspension was aspirated and nichoids covered with 400 uL of citrate solution (Sodium citrate dihydrate 0.015 M and KCl 0.135 M). Upon gentle resuspension, cells were incubated for 15 min in a 5% CO₂ humidified atmosphere at 37° C. Next, cells were collected and centrifuged at 300 g×10 min.

Mice. NOD-SCID-IL2Rg_/_ (NSG) mice were purchased from The Jackson Laboratory and maintained in specific-pathogen-free (SPF) conditions. The procedures involving animals were designed and performed with the approval of the Animal Care and Use Committee of the San Raffaele Hospital (IACUC #1165) and communicated to the Ministry of Health and local authorities according to Italian law.

Gene editing of human HSPCs and analyses. After 3 days of stimulation, cells were washed with PBS and electroporated using P3 Primary Cell 4D-Nucleofector X Kit and program EO-100 (Lonza). Cells were electroporated with 1.25-2.5 mM of RNPs as indicated performing the editing of the AAVS1 locus. Transduction with AAV6 was performed at doses of 1 or 1×10⁴ vg/cell 15 minutes after electroporation. Gene editing efficiency was measured from cultured cells in vitro 96 hours after electroporation for CB and mPB-derived HSPCs by flow cytometry measuring the percentage of cells expressing the GFP marker, or by digital droplet PCR analysis designing primers and probe on the junction between the vector sequence and the targeted locus and on control sequences utilized as normalizer as previously described.

Colony-forming unit cell assay. CFU-C assay was performed at the indicated, plating 800 cells in methylcellulose-based medium (MethoCult H4434, StemCell Technologies) supplemented with 100 IU/ml penicillin and 100 mg/ml streptomycin. Two weeks after plating, colonies were counted in blinded fashion, and erythroid, myeloid, and mixed colonies were identified according to morphological criteria.

CD34+ HSPC xenotransplantation studies in NSG mice. For transplantation, 1.5×10⁵ CD34+ cells were injected intravenously into NSG mice after sublethal irradiation (150-180 cGy) at the indicated timepoint. Sample size was determined by the total number of available treated cells. Mice were randomly attributed to each experimental group. Human CD45+ cell engraftment was monitored by serial collection of blood from the mouse tail.

Flow cytometry. Immunophenotypic analyses were performed on the fluorescence activated cell sorting (FACS) Canto II (BD Pharmingen). From 0.5×10⁵ to 2×10⁵ cells (either from culture or mouse samples) were analysed by flow cytometry. Ex vivo treated cells were stained for 15 min at 4° C. with CD34-PE, CD133-PEcy7 and CD90-APC antibodies, while peripheral blood-derived, bone marrow-derived, or spleen-derived cells with CD45-APCCy7, CD34-PECy7, CD19-PE, CD3-APC, CD13 BV421 in a final volume of 50 μl and then washed with DPBS+2% heat-inactivated FBS. The Live/Dead Fixable Dead Cell Stain Kit (Thermo Fisher) or 7-aminoactinomycin D (Sigma Aldrich)/Annexin V Pacific blue staining were included during sample preparation according to the manufacturer's instructions to identify dead cells. Apoptosis analysis was performed as previously described (Schiroli et al., 2019, Cell Stem Cell 24: 551-565). Proliferation analyses were performed with CellTracker Violet BMQC Dye (Thermo Scientific) according to manufacturer's instruction. Single-stained and fluorescence-minus-one-stained cells were used as controls. Data were analysed with FlowJo software v. 10.8.1.

Molecular analyses. For molecular analyses, gDNA was isolated with QIAamp DNA Micro Kit (QIAGEN) according to the manufacturer's instructions.

For HDR digital droplet PCR (ddPCR) analysis, 5-30 ng of gDNA were analysed using the QX200 Droplet Digital PCR System (Bio-Rad) according to the manufacturer's instructions. HDR ddPCR primers and probes were designed on the junction between the vector sequence and the targeted locus. Human TTC5 (Bio-Rad) was used for normalization.

Immunofluorescence Analysis. Multitest slides (15 well, MP Biomedicals) were treated for 20′ with Poly-L-lysine solution (Sigma-Aldrich) at 1 mg/ml concentration. After two washes with DPBS solution, approximately 3-5×10⁴ cells were seeded on covers for 20′ and fixed with 4% paraformaldehyde (Santa Cruz Biotechnology) for other 20′. Cells were then permeabilized with 0.5% Triton X-100. After blocking with 0.5% BSA and 0.2% fish gelatine in DPBS, cells were probed with the indicated primary antibodies. After primary antibodies incubation (Anti-phospho Histone H2A.X (Ser139) Antibody, clone JBW301, Merck; pRPA (S33) Antibody, polyclonal, Bethyl Laboratories), cells were washed three times with DPBS and incubated with Alexa 568- and 647-labeled secondary antibodies (Invitrogen). Nuclear DNA was stained with DAPI at 0.2 mg/ml concentration (Sigma-Aldrich) and covers were mounted with Aqua-Poly/Mount solution (Polysciences. Inc.) on glass slides (Bio-Optica). Fluorescent images were acquired using Leica SP5 Confocal microscopes. Quantification of DDR foci in immunofluorescence images was conducted using ImageJ.

Alkaline comet assay. 3×10³ cells per condition were mixed with molten Comet LMAgarose (Trevigen, MD) at a ratio of 1:10 (v/v) and immediately pipetted onto CometSlides (Trevigen, MD) and placed at +4° C. for 30′. Once solidified, the slides were immersed in prechilled Lysis Solution (Trevigen, MD) for 1 h at +4° C. Following lysis, slides were immersed in freshly prepared Alkaline Unwinding Solution pH>13 (300 mM NaOH, 1 mM EDTA) for 1 h at +4° C. and then electrophoresed in alkaline electrophoresis solution pH>13 (300 mM NaOH, 1 mM EDTA) at 1V/cm (21V) for 30 min. Slides were washed twice in ddH2O and fixed in 70% ethanol for 5 min. Comets were stained with SYBR Safe for 30′ at RT. All steps were conducted in the dark to prevent additional DNA damage. Comets were analysed using a Nikon Eclipse E600 microscope and a Nikon-DS-RI2 camera. Up to 100 nuclei for each individual donor were analysed with CaspLab-Comet assay software project to determine “Olive Tail Moments” of individual nuclei.

### Quantification and Statistical Analysis

Data were expressed as means±SEM or dot plots with median values indicated as a line. Inferential techniques were carried out whenever appropriate sample size was available, otherwise descriptive statistics are reported. Percentage values were transformed into a log-odds scale to perform parametric statistical analyses. Assumptions for the correct application of standard parametric procedures were checked (e.g., normality of the data). t test for paired data was applied to compare dependent observations.

### EXAMPLE 1

### Growth of HSPC on Nichoids

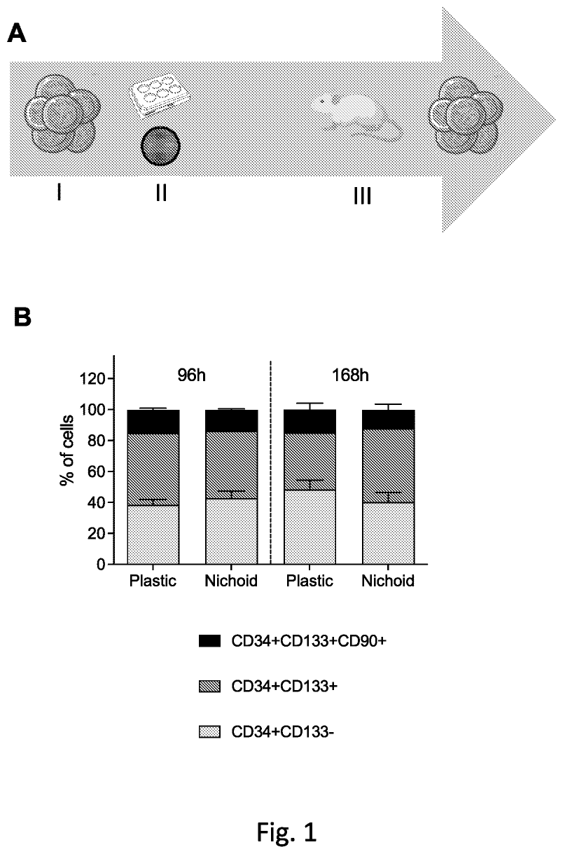

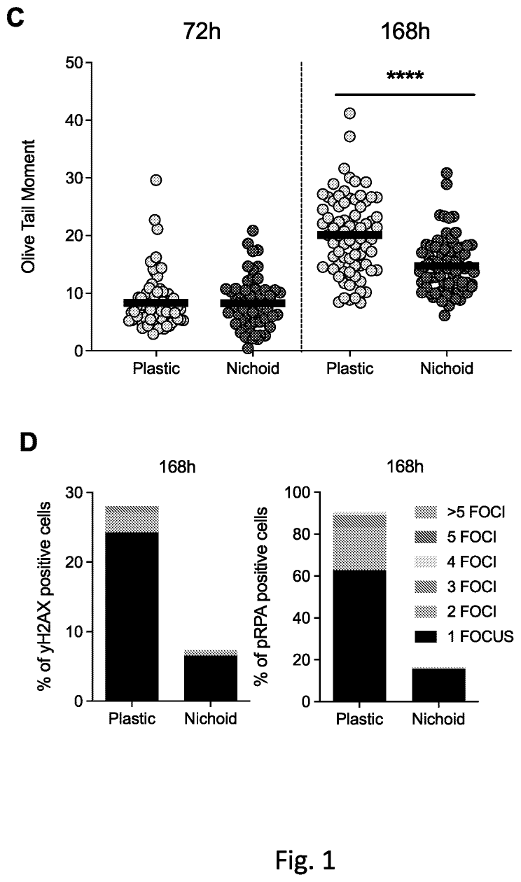

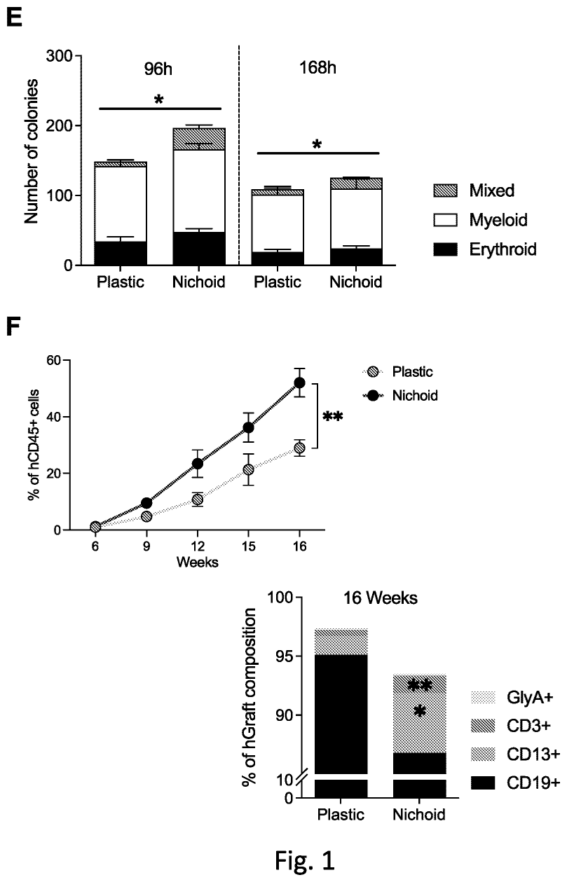

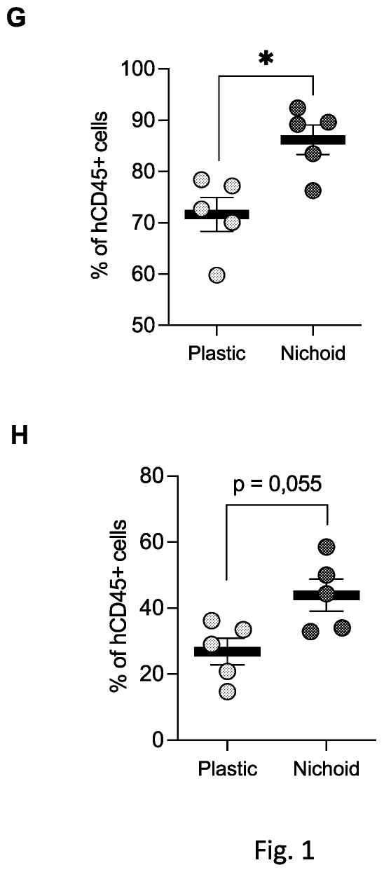

HSPCs were seeded either on standard culture wells, made of plastic material, or on 3D nichoids scaffolds, and analysed at different times of culture (FIG. 1A). Subset composition analysis did not indicate relevant differences in HSPC differentiation patterns upon nichoid culture (FIG. 1 B). However, three-dimensional culture mitigates physical DNA damage accumulation over time, as observed by alkaline comet assay that allows visualization of both single- and double-strand breaks (FIG. 1 C). Consistent with this result, reduced foci formation of γH2Ax and pRPA DDR sensors were reported in the nichoid condition (FIG. 1 D). Moreover, HSPC in vitro clonogenic capacity was improved upon three-dimensional culture. Surprisingly, the increased colony output was particularly associated with a higher number of mixed colonies (CFU-GEMM), the progeny of more primitive and multipotent HSCs (Carow, Hangoc and Broxmeyer, 1993, Blood 81: 942-949) (FIG. 1E). Finally, plastic or nichoid-cultured CD34+ cells were transplanted into NOD Prkdescid Il2rg−/− (NSG) mice and HSPC in vivo repopulating potential was followed by performing serological analysis at different time points post-injection. The percentage of human CD45+ cells in the peripheral blood of mice transplanted with HSPCs upon nichoid in vitro culture was effectively increased up to 16 weeks, with improved multi-lineage output of both CD3+ and CD13+ cells within the human graft (FIG. 1 F). Moreover, higher human engraftment was found in the nichoid group also across the other hematopoietic organs of the recipient mice, including the bone marrow (FIG. 1 G) and the spleen (FIG. 1 H), indicating that tridimensional cues and geometrical constraints are crucial to preserve HSPC biological properties.

### EXAMPLE 2

### Growth of HSPC on Nichoids Prior to the Gene-Editing Procedure

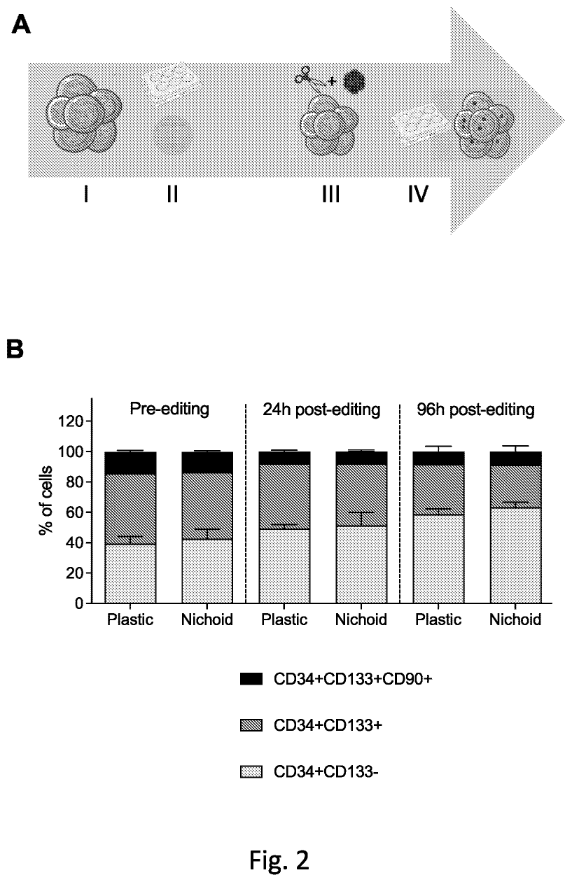

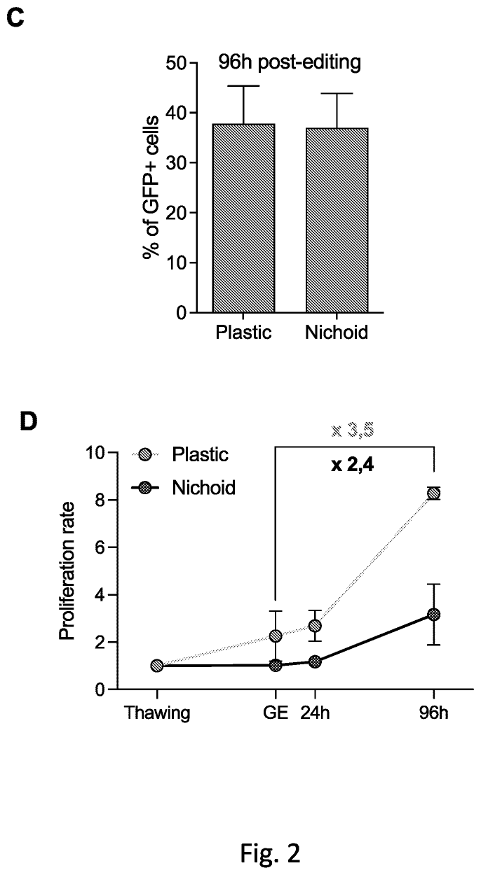

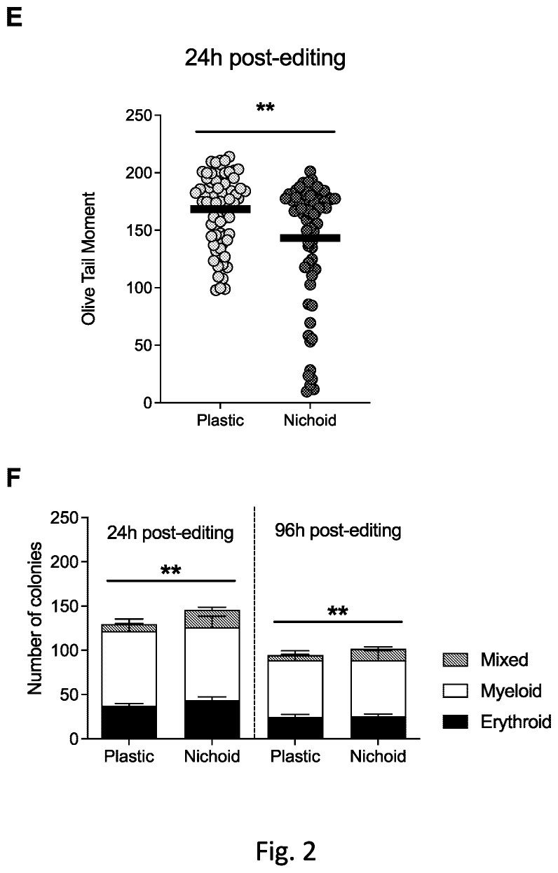

Given these results obtained in Example 1, nichoids were exploited during the ex vivo culture required for HSPC genetic engineering. Thus, at the moment of thawing HSPCs were seeded either on standard culture wells or on nichoids. After three days of cytokine stimulation, gene editing (GE) was performed by electroporation of Cas9 RNPs in the presence of an AAV6 vector to achieve homology-directed repair (HDR)-mediated insertion of a PGK.GFP reporter cassette within the AAVS1 locus. Upon GE, cells were replated on standard culture wells and analyzed at the indicated time points (FIG. 2A). Flow cytometry analyses revealed similar culture composition and editing efficiencies between the different culture conditions (FIG. 2 B, C). Moreover, reduced physical DNA damage levels were reported in nichoid-cultured HSPCs upon genetic manipulation (FIG. 2 D, E). Finally, the in vitro functionality of edited HSPCs was enhanced upon three-dimensional culture prior to GE (FIG. 2 F).

Overall, the results indicate that synthetic three-dimensional niches may represent the best culture tool for a plethora of engineered stem cell-based therapeutic applications.

### EXAMPLE 3

### Gene-Edited HSPCs Pre-Cultured on Nichoids Show Superior Engraftment In Vivo

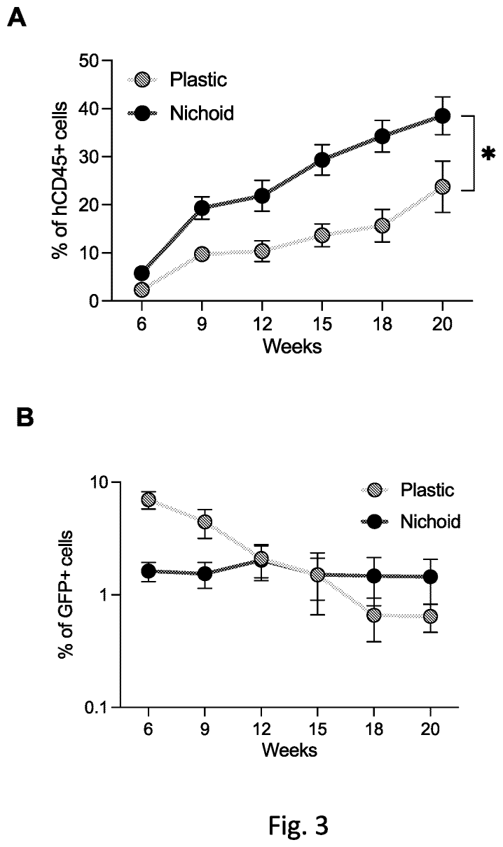

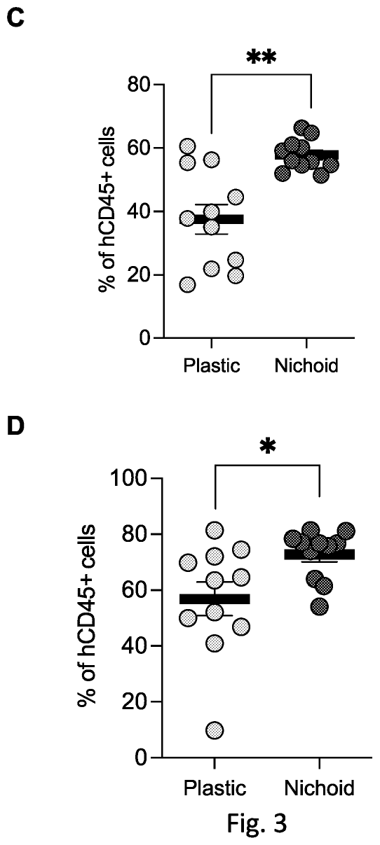

Given the positive results obtained in Examples 1 and 2, CD34+ cells, gene-edited upon plastic or nichoid pre-culture, were transplanted into NSG mice at 24 h post-editing and HSPC engraftment was monitored at different time points post-injection. Higher human chimerism was reported in the peripheral blood of mice transplanted with nichoid-cultured HSPCs (FIG. 3 A), with more stable engraftment of HDR-edited cells (GFP+), which conversely was drastically reduced overtime in the control group (FIG. 3 B). In addition, a higher percentage of human CD45+ cells was present in the bone marrow and spleen of NSG mice from the nichoid condition (FIG. 3 C, D), further confirming the beneficial effects of 3D culture for the preservation of HSPC functionality during ex vivo manipulation for gene-editing applications.

### EXAMPLE 4

### Nichoids Improve HSPC Functionality in Various Clinically Relevant Settings

To test 3D culture in different gene therapy settings, an optimized protocol of lentiviral transduction (Gentner et al., 2021), consisting of a total of 38 h ex vivo culture, was applied.

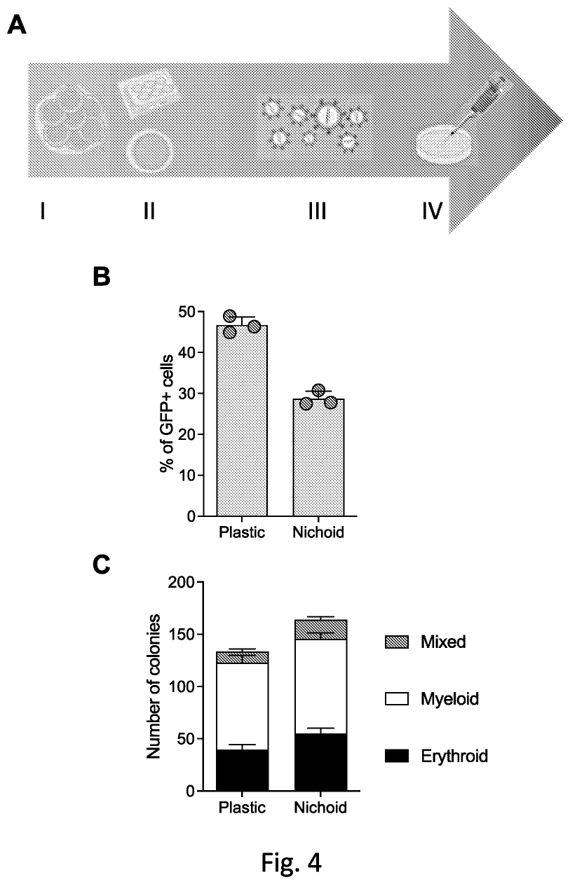

Thus, HSPCs were seeded either on standard culture wells (plastic) or on nichoids, and after 24 h from thawing. 2 h pre-stimulation with PGE2 was performed, followed by administration of a lentiviral vector encoding for a PGK.GFP reporter cassette. After 14 h post-transduction, the cells were washed and collected for in vitro analyses (FIG. 4A). Gene transfer efficiency was slightly reduced upon transduction in 3D culture (FIG. 4 B); however, a substantial increase in the clonogenic potential of the nichoid condition was observed, attributable to the expansion of the mixed colony output (FIG. 4 C).

In addition, nichoids were tested in the context of human HSPC expansion, which is particularly relevant for some genetic disorders in which only a limited number of HSPCs can be retrieved from patients.

Thus, it was employed an expansion protocol of 8 days of ex vivo culture, including lentiviral transduction of a PGK.GFP reporter in the presence of the transduction enhancer Cyclosporin

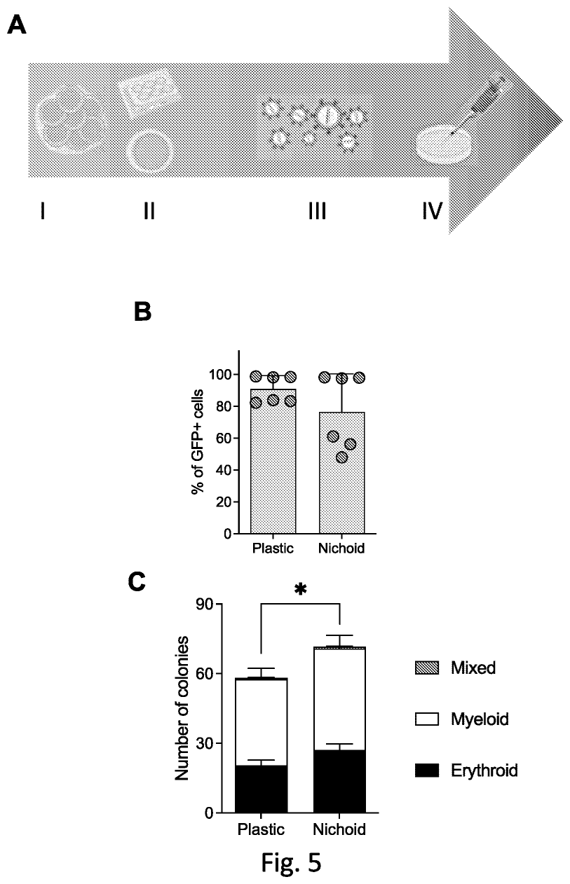

H (CsH) (Petrillo et al., 2018) (FIG. 5A). Hence, gene transfer efficiency was assessed by FACS analysis at the endpoint of the experiment. A slightly lower percentage of GFP+ cells was observed in the nichoid condition CsH (FIG. 5 B). Nevertheless, nichoids ameliorated HSPC colony output (FIG. 5 C), indicating that 3D interactions advantageously preserve HSPC functionality in multiple settings of genetic engineering.

### EXAMPLE 5

### Culture on Nichoids Outperformed Current Clinical-Grade Cell Culture Bags In Vitro

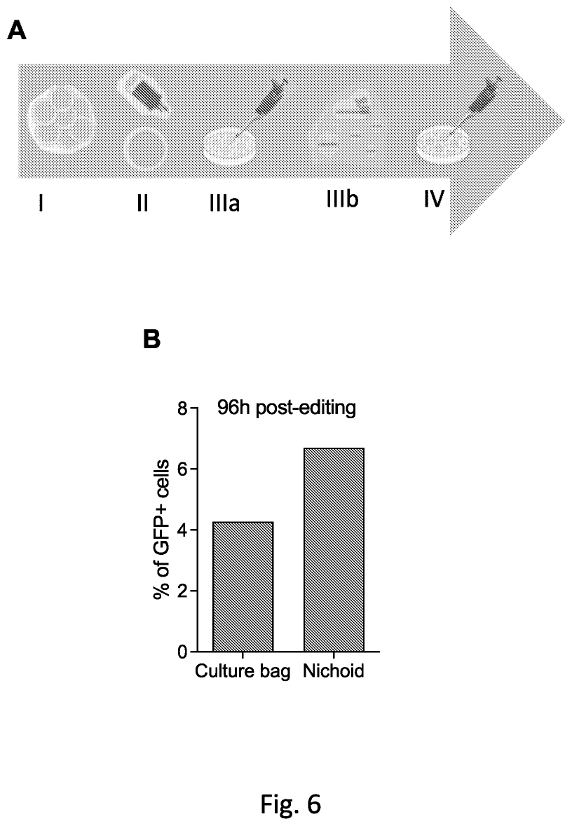

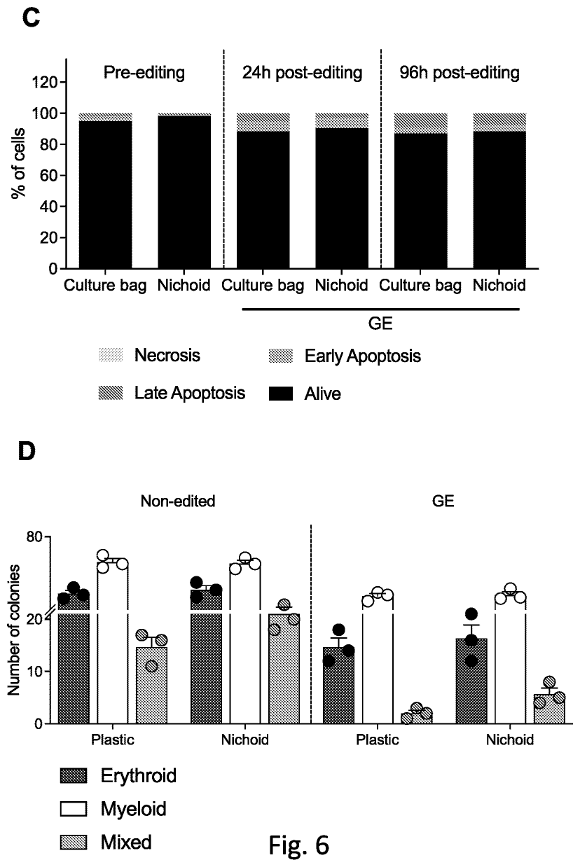

Finally, nichoids were compared with cell culture bags, which are the current gold standard for culturing HSPCs during ex vivo manipulation for gene therapy clinical application. HSPCs were then seeded either on a cell culture bag or on nichoids, and after three days of cytokine stimulation, cells were collected for downstream analyses before performing Cas9/AAV6-mediated gene editing (GE). Upon GE, cells were replated on standard culture wells and analyzed at the indicated time points (FIG. 6A). FACS analyses revealed a similar GE efficiency between the two conditions (FIG. 6 B) and comparable levels of cell viability assessed by Annexin V and 7-AAD staining (FIG. 6 C).

Regardless, HSPC mixed colony output and in vitro clonogenic potential were enhanced upon nichoid culture both in non-edited and GE-cells (FIG. 6 D), suggesting that 3D scaffolds represent the best culture tool for a plethora of HSPC-based therapeutic applications.

1. An ex vivo method of engineering cells, comprising:
genetically modifying the cells ex vivo and
culturing the cells on a three-dimensional (3D) scaffold.

2. The ex vivo method of claim 1, wherein the cells are stem cells or T cells.

3. The ex vivo method of claim 1, wherein the cells are hematopoietic stem and/or progenitor cells.

4. The ex vivo method of claim 3, wherein the cells are hematopoietic stem and/or progenitor CD34+ cells.

5. The ex vivo method of claim 1, wherein the cells are mesenchymal stem cells.

6-7. (canceled)

8. The ex vivo method of claim 1, wherein the cells are genetically modified by gene editing.

9. The ex vivo method of claim 1, wherein the cells are genetically modified by gene editing and wherein gene editing comprises transfecting or transducing the cell to express one or more of: zinc-finger nucleases, transcription activator like effector nucleases (TALENs), CRISPR system.

10. The ex vivo method of claim 1, wherein the cells are genetically modified by gene transfer.

11. The ex vivo method of claim 1, wherein the step(s) of genetically modifying the cells ex vivo comprises transducing the cells with a viral vector.

12. The ex vivo method of claim 1, wherein the step(s) of genetically modifying the cells ex vivo comprises transducing the cells with a retroviral vector.

13. The ex vivo method of claim 1, wherein the step(s) of genetically modifying the cells ex vivo comprises transducing the cells with an adeno-associated vector (AAV).

14. The ex vivo method of claim 1, wherein the step(s) of genetically modifying the cells ex vivo comprises transducing the cells with a lentiviral vector, or an integration-defective lentiviral vector (IDLV).

15. The ex vivo method of claim 1, wherein the step(s) of genetically modifying the cells ex vivo includes stimulation of cells by a mix of cytokines and/or includes addition of a viral transduction enhancer to the cell culture.

16. (canceled)

17. The ex vivo method of claim 1, wherein the step of culturing the cells on the 3D scaffold precedes the step(s) of genetically modifying the cells ex vivo.

18. The ex vivo method of claim 1, wherein the step of culturing the cells on the 3D scaffold precedes the step(s) of genetically modifying the cells ex vivo, and wherein the step of genetically modifying the cells ex vivo is carried out 1 to 4 days after seeding the cells on a three-dimensional (3D) scaffold.

19. The ex vivo method of claim 1, comprising a step of culturing the cells on the 3D scaffold for at least 1, at least 2, at least 3 days, or at least 4 days, followed by a step of genetically modifying the cells, or comprising a step of culturing the cells on the 3D scaffold for 1 to 8 days after the step of genetically modifying the cells, preferably for 2 to 6 days, most preferably for 3 to 5 days, after the step of genetically modifying the cells.

20-21. (canceled)

22. The ex vivo method of claim 1, wherein the step of culturing the cells on the 3D scaffold begins with seeding the cells on a 3D scaffold and ends with collecting the engineered cells, preferably wherein said step of culturing the cells on the 3D scaffold is carried out for 2 to 8 days, more preferably for 4 to 8 days, most preferably for about 8 days.

23. The ex vivo method of claim 1 wherein the scaffold is, or is connected to, an implant.

24. A population of ex vivo engineered cells obtained by the method of claim 1.

25-26. (canceled)

27. A method of treating cancer, an immune disorder, a bacterial or viral infection, a genetic disease, a blood disease, a lysosomal storage disease, β-thalassemia, Fanconi anemia, bone marrow failure disease, sickle cell disease, osteopetrosis, chronic granulomatous disease, metachromatic leukodystrophy, or mucopolysaccharidosis in a subject in need thereof, said method comprising
administering to said subject an effective amount of a population of ex vivo engineered cells obtained by the method of claim 1.
